(19) United States
(12) Patent Application Publication
(10) Pub. No.: US 2024/0353271 A1
(43) Pub. Date: Oct. 24, 2024

(54) TEMPERATURE MEASUREMENT DEVICE

(71) Applicant: Nippon Telegraph and Telephone Corporation, Tokyo (JP)

(72) Inventors: Yujiro Tanaka, Tokyo (JP); Daichi Matsunaga, Tokyo (JP)

(21) Appl. No.: 18/701,134

(22) PCT Filed: Dec. 16, 2021
(86) PCT No.: PCT/JP2021/046505

§ 371 Date: Apr. 12, 2024

(51) Int. Cl. G01K 13/20; G01K 1/024; G01K 1/16; G01K 3/00; G01K 17/00

(52) U.S. Cl. CPC G01K 13/20; G01K 1/024; G01K 1/16; G01K 3/005; G01K 17/00

(57) ABSTRACT

An embodiment is a temperature measuring device including a sensor configured to measure a magnitude of a heat flow transmitted from a living body, an electronic circuit configured to calculate an internal temperature of the living body, on the basis of the magnitude of the heat flow measured by the sensor, wherein the sensor includes a heat conductor of a hollow structure disposed so that a peripheral edge part is configured to be in contact with the living body, a first covering material configured to fill a space between the living body and the heat conductor, a detector on the first covering material configured to measure the magnitude of the heat flow transmitted from the living body, and a second covering material covering the heat conductor.

### CROSS-REFERENCE TO RELATED APPLICATIONS

This application is a national phase entry of PCT Application No. PCT/JP2021/046505, filed on Dec. 16, 2021, which application is hereby incorporated herein by reference.

### TECHNICAL FIELD

The present invention relates to a temperature measuring device that measures an internal temperature of a living body non-invasively and accurately.

### BACKGROUND

A technique for non-invasively measuring the deep body temperature of a living body is known in the related art. For example, NPL 1 discloses a technique for estimating the body temperature of the deep portion of the living body by assuming a pseudo one-dimensional model of outside air and the living body.

The technique disclosed in NPL 1 estimates a deep body temperature T_{body} of a living body 100, by assuming a one-dimensional model of heat transfer between the living body 100 and a sensor 101, as shown in FIG. 17. In FIG. 1, Tₐᵢᵣ is a temperature of outside air, H_{signal} is a heat flux flowing into the sensor 101, Rₐᵢᵣ is heat resistance when the heat flux H_{signal} moves to the outside air, Tₛₖᵢₙ is a temperature of a skin surface of the living body 100 measured by the sensor 101, and Tₜ is a temperature of an upper surface of the sensor 101 on a side opposite to a surface that is in contact with the living body 100. The deep body temperature T_{body} of the living body 100 can be estimated, using equation (1).

$${T}_{body} = {T}_{skin} + {R}_{sensor} \times {H}_{signal} \qquad ( 1 )$$

A proportionality coefficient Rₛₑₙₛₒᵣ can be obtained as following equation, by substituting a tympanic membrane temperature measured by a tympanic membrane thermometer at the start of measurement or during measurement or a rectum temperature measured by a rectum thermometer or axillary temperature measured by thermometer, into equation (1) as a deep body temperature T_{body} (reference temperature).

$${R}_{sensor} = ( {T}_{body} - {T}_{skin} ) / ( {T}_{skin} - {T}_{t} ) \qquad ( 2 )$$

Therefore, by measuring the temperature Tₛₖᵢₙ of the skin surface and the heat flux H_{signal} flowing into the sensor 101, the deep body temperature T_{body} of the living body can be estimated by equation (1).

However, when a one-dimensional model is assumed as a heat transfer model of the living body 100 as in the technique disclosed in the NPL 1, as shown in FIG. 17, due to the generation of airflows and changes in the outside air temperature, a heat flux H_{Leak} occurs in which the heat that should flow into the sensor 101 deviates from the original flow and moves in a lateral direction. When the thermal resistance between the sensor 101 and the outside air changes due to airflows or an outside air temperature, and a heat flux H_{Leak} deviating from the sensor 101 occurs, the heat flux that should be measured H_{signal} decreases to H′signal.

As described above, when wind blows against the living body 100 or the outside air temperature changes, the one-dimensional model of heat transfer is not established. Therefore, in the technique of the related art, there was a problem that an error occurred in estimation of the deep body temperature T_{body} due to generation of wind and a change in the outside air temperature.

### CITATION LIST

### Non Patent Literature

[NPL 1] H.-C. Gunga, et al., “The Double Sensor. A non-invasive device to continuously monitor core temperature in humans on earth and in space”, Respiratory Physiology & Neurobiology, 2009

### SUMMARY

### Technical Problem

The present invention was made to solve the foregoing problems, and an object thereof is to provide a temperature measuring device that can suppress changes in thermal resistance between the sensor and the outside air, and accurately measure the internal temperature of the living body.

### Solution to Problem

A temperature measuring device of the present invention includes a sensor unit configured to measure a magnitude of a heat flow transmitted from a living body, and an electronic circuit unit configured to calculate an internal temperature of the living body, on the basis of the magnitude of the heat flow measured by the sensor unit. The sensor unit includes a heat conductor of a hollow structure disposed so that a peripheral edge part is in contact with the living body, a first covering material disposed to fill a space between the living body and the heat conductor, a detection unit provided on the first covering material to measure the magnitude of the heat flow transmitted from the living body, and a second covering material disposed to cover the heat conductor.

In one configuration example of the temperature measuring device according to the present invention, the electronic circuit unit is provided inside the second covering material beside the sensor unit.

In one configuration example of the temperature measuring device according to the present invention, the electronic circuit unit is provided inside the second covering material on the sensor unit.

One configuration example of the temperature measuring device of the present invention further includes a housing provided to cover the second covering material on the outer side.

Also, in one configuration example of the temperature measuring device of the present invention, the detection unit includes a first temperature sensor provided on a surface of the first covering material facing the living body and configured to measure a temperature of a surface of the living body, and a second temperature sensor configured to measure a temperature inside the first covering material immediately above the first temperature sensor. The electronic circuit unit calculates an internal temperature of the living body on the basis of measurement results of the first and second temperature sensors.

In one configuration example of the temperature measuring device of the present invention, the detection unit includes a temperature sensor provided on the surface of the first covering material facing the living body and configured to measure the temperature of the surface of the living body, and a heat flux sensor provided on the surface of the first covering material facing the living body and configured to measure the heat flux flowing from the living body into the sensor unit. The electronic circuit unit calculates an internal temperature of the living body on the basis of measurement results of the temperature sensor and the heat flux sensor.

### Advantageous Effects of Invention

According to the present invention, since a heat conductor is provided at a position away from a detection part, heat of a living body is transported through the heat conductor and a temperature of an upper part of the detection part is raised, thereby suppressing a lateral heat flux deviating from a pseudo one-dimensional model in the outside air and the living body, the internal temperature of the living body can be accurately measured, even when the temperature around the sensor unit is changed or wind is generated.

### BRIEF DESCRIPTION OF THE DRAWINGS

FIG. 1 is a diagram showing a configuration of a temperature measuring device according to a first example of the present invention.

FIG. 2 is an external view of the temperature measuring device according to the first example of the present invention.

FIG. 3 is a partially cutaway perspective cross-sectional view of a heat conductor according to the first example of the present invention.

FIG. 4 is a flowchart for explaining the operation of the temperature measuring device according to the first example of the present invention.

FIGS. 5A to 5D are diagrams showing an example of attaching the temperature measuring device according to the first example of the present invention to the living body.

FIGS. 6A to 6C are diagrams showing an example of attaching the temperature measuring device according to the first example of the present invention to the living body.

FIG. 7 is a diagram showing a deep body temperature estimated by the temperature measuring device according to the first example of the present invention and the tympanic membrane temperature measured by the tympanic membrane thermometer.

FIG. 8 is a diagram showing temporal changes in the deep body temperature estimated by the temperature measuring device according to the first example of the present invention and the tympanic membrane temperature measured by the tympanic membrane thermometer.

FIG. 9 is a diagram showing a configuration of a temperature measuring device according to a second example of the present invention.

FIG. 10 is an external view of the temperature measuring device according to the second example of the present invention.

FIG. 11 is a flowchart for explaining the operation of the temperature measuring device according to the second example of the present invention.

FIG. 12 is a diagram showing a configuration of a temperature measuring device according to a third example of the present invention.

FIG. 13 is an external view of the temperature measuring device according to the third example of the present invention.

FIG. 14 is a diagram showing a configuration of a temperature measuring device according to a fourth example of the present invention.

FIG. 15 is an external view of the temperature measuring device according to the fourth example of the present invention.

FIG. 16 is a block diagram showing a configuration example of a computer that implements the temperature measuring device according to the first to fourth examples of the present invention.

FIG. 17 is a diagram showing a thermal equivalent circuit model of the living body and the sensor.

### DETAILED DESCRIPTION OF ILLUSTRATIVE EMBODIMENTS

### First Example

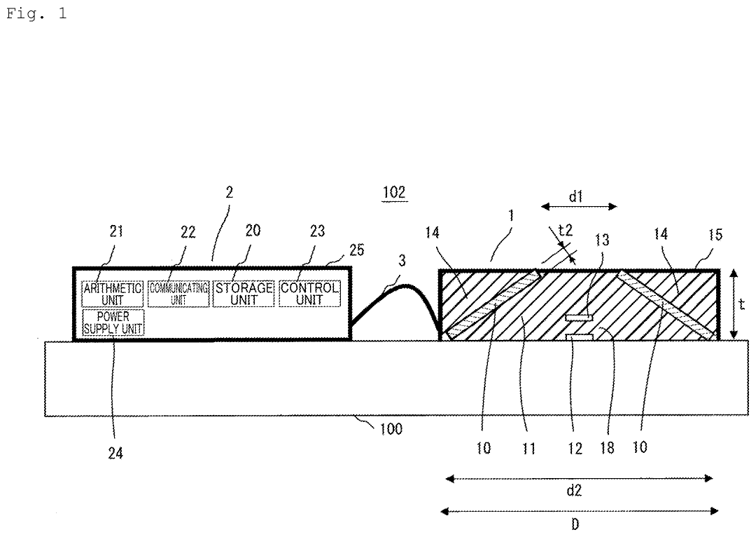

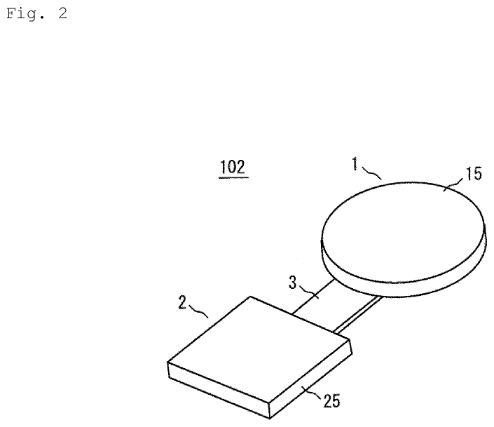

Referring to the drawings, a description will be given of examples of the present invention. FIG. 1 is a diagram showing the configuration of a temperature measuring device according to a first embodiment of the present invention, and FIG. 2 is an external view of the temperature measuring device. A temperature measuring device 102 includes a sensor unit 1 which measures the magnitude of a heat flow transmitted from the living body 100, and an electronic circuit unit 2 which calculates a deep body temperature T_{body} (internal temperature) of the living body 100 on the basis of a measured magnitude of the heat flow.

The sensor unit 1 includes a heat conductor 10 of a hollow structure which is disposed so that a peripheral edge part is in contact with the living body 100 and transports a heat flux from the living body 100 to an upper part of the sensor unit 1, a covering material 11 disposed to fill a space between the living body 100 and the heat conductor 10, a temperature sensor 12 which is provided on a surface of the covering material 11 facing the living body 100 and measures a temperature Tₛₖᵢₙ of the skin surface of the living body 100, a temperature sensor 13 which measures a temperature Tₜ inside the covering material 11 just above the temperature sensor 12, a covering material 14 disposed to cover the heat conductor 10, and a housing 15 which houses the heat conductor 10, the covering materials 11 and 14, and the temperature sensors 12 and 13. The temperature sensors 12 and 13 constitute a detection unit 18 which measures the magnitude of the heat flow.

The electronic circuit unit 2 includes a storage unit 20 for storing data, an arithmetic unit 21 which calculates a deep body temperature T_{body} of the living body 100 on the basis of measurement results of the temperature sensors 12 and 13, a communication unit 22 which transmits data of the deep body temperature T_{body} to an external terminal, a control unit 23 which controls reading/writing and communication of data to the storage unit 20, a power supply unit 24 which supplies power to the storage unit 20, the arithmetic unit 21, the communication unit 22, and the control unit 23, and a housing 25 which houses the storage unit 20, the arithmetic unit 21, the communication unit 22, the control unit 23, and the power supply unit 24.

The sensor unit 1 is mounted so that the covering material 11 and the heat conductor 10 come into contact with the skin of the living body 100. It is desirable to mount the sensor unit 1 on the living body 100, for example, using a double-sided tape or silicone rubber excellent in biocompatibility. For example, a thermistor, a thermocouple, a platinum resistor, an integrated circuit (IC) temperature sensor, or the like can be used as the temperature sensors 12 and 13.

The temperature sensor 13 is disposed immediately above the temperature sensor 12. When the interval between the temperature sensors 12 and 13 changes during measurement, a proportionality coefficient Rₛₑₙₛₒᵣ changes, and an error occurs in estimation of the deep body temperature T_{body} of the living body 100. Thus, the temperature sensors 12 and 13 are held, using the covering material 11. In consideration of heat leakage, it is necessary to use a material having a thermal conductivity smaller than that of the heat conductor 10 as the covering material 11, and it is desirable to use a material having a thermal conductivity similar to that of the living body 100 (0.2 to 0.5 W/m²).

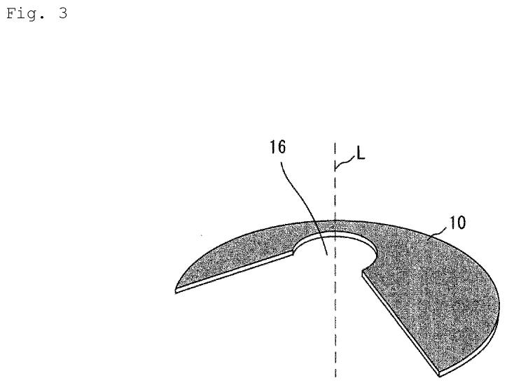

Further, the covering material 11 holds a relative positional relationship between the heat conductor 10 and the temperature sensors 12 and 13. FIG. 3 is a partially cutaway perspective cross-sectional view of the heat conductor 10. The heat conductor 10 has a frustum shape in which an area of a top surface separated from the living body 100 is smaller than an area of a bottom surface on the living body 100 side. It is preferred that the material constituting the heat conductor 10 have a high thermal conductivity to efficiently transport a heat flux. For example, the heat conductor 10 can be configured, using a metal such as aluminum.

The material of the heat conductor 10 may be a material obtained by knitting a resin containing metal, graphite, carbon nanotube or the like, or a metal fiber into a predetermined shape, in addition to metal. Further, by orienting graphite or carbon nanotubes in the plane of the sheet-like resin, it is possible to realize the heat conductor 10 having thermal conductivity anisotropy and flexibility in which thermal conductivity in an in-plane direction perpendicular to a thickness direction is higher than thermal conductivity in the thickness direction. Further, a liquid such as graphite, carbon nanotube or grease containing metal may be used as the heat conductor 10. As shown in FIGS. 1 and 3 a through-hole 16 may be formed on the top surface of the heat conductor 10.

When the heat conductor 10 is sufficiently large with respect to the temperature sensors 12 and 13, because the peripheral edge part of the heat conductor 10 that is in contact with the living body 100 is disposed at a position sufficiently separated from the temperature sensors 12 and 13, a heat flux from the living body 100 is collected by the heat conductor 10 outside the temperature sensors 12 and 13 and transported to the top surface of the heat conductor 10. Thus, the heat conductor 10 performs a function of efficiently transporting the heat flux from the living body 100 upward outside the temperature sensors 12 and 13, thereby suppressing the heat flux that escapes from the temperature sensors 12 and 13 and flows out to the outside air. In the heat conductor 10, the effects of suppressing the heat flux from deviating from the temperature sensors 12 and 13 and flowing out to the outside air is highest at a position near the center line (L of FIG. 3). Therefore, it is desirable to dispose the temperature sensors 12 and 13 near the center line L of the heat conductor 10.

As described above, the through-hole 16 may be formed on the top surface of the heat conductor 10. By adjusting the size of the through-hole 16 appropriately, it is possible to adjust the depth of measurement in the case of measuring the deep body temperature T_{body} of the living body 100. However, the provision of the through-hole 16 in the heat conductor 10 is not an essential component requirement of the present invention.

As the material of the covering material 14, the same material as that of the covering material 11 can be used. The same material as the covering materials 11 and 14 may be used as the materials for the housings 15 and 25. Most of resin materials can be used as the covering materials 11, 14 and the housings 15 and 25.

If a flexible material is used as the covering materials 11 and 14, the heat conductor 10 and the housing 15, the material can be deformed according to the complicated shape of the living body 100. Similarly, if the electronic circuit unit 2 is mounted on a flexible substrate such as polyimide and a flexible material is used as the housing 25, the material can be deformed according to the shape of the living body 100. Therefore, the sensor unit 1 and the electronic circuit unit 2 can be easily mounted on the living body 100. The wearing feeling to the living body 100 can be improved.

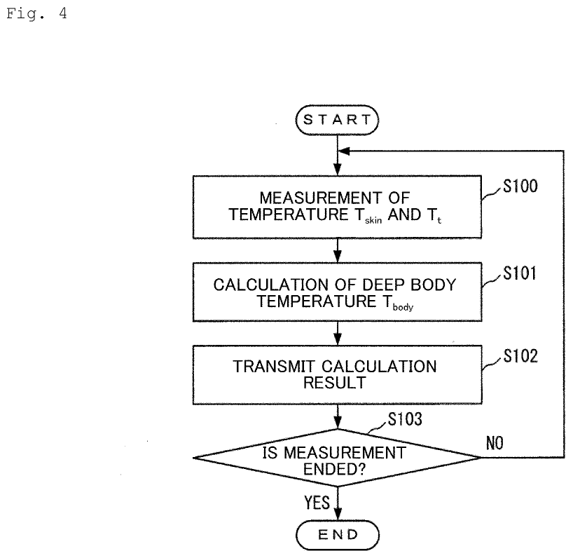

The temperature sensors 12 and 13 and the electronic circuit unit 2 are connected by a wiring 3. FIG. 4 is a flowchart for explaining the operation of the temperature measuring device 102 of the present embodiment. The temperature sensor 12 measures the temperature Tₛₖᵢₙ of the skin surface of the living body 100. The temperature sensor 13 measures a temperature Tₜ of the inside of the covering material 11 at a position away from the living body 100 (step S100 of FIG. 4). Measured data of the temperature sensors 12 and 13 is stored in the storage unit 20 once.

The proportional coefficient Rₛₑₙₛₒᵣ is stored in the storage unit 20 in advance. The arithmetic unit 21 calculates the deep body temperature T_{body} of the living body 100 by, for example, equation (3) on the basis of the temperatures Tₛₖᵢₙ and Tₜ and the proportional coefficient Rₛₑₙₛₒᵣ (step S101 of FIG. 4).

$${T}_{body} = {T}_{skin} + {R}_{sensor} \times ( {T}_{skin} - {T}_{t} ) \qquad ( 3 )$$

The calculation of Tₛₖᵢₙ−Tₜ as in equation (3) corresponds to the calculation of the heat flux H_{signal} of equation (1).

The communication unit 22 transmits data of the deep body temperature T_{body} to an external terminal, for example, a personal computer (PC) or a smart phone (step S102 of FIG. 4). The external terminal displays the value of deep body temperature T_{body} received from the temperature measuring device 102.

The temperature measuring device 102 executes the processing of steps S100 to S102 at every fixed time, for example, until a user instructs the end of measurement (YES in step S103 of FIG. 4).

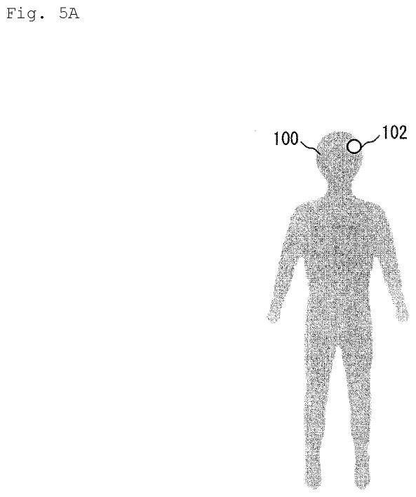

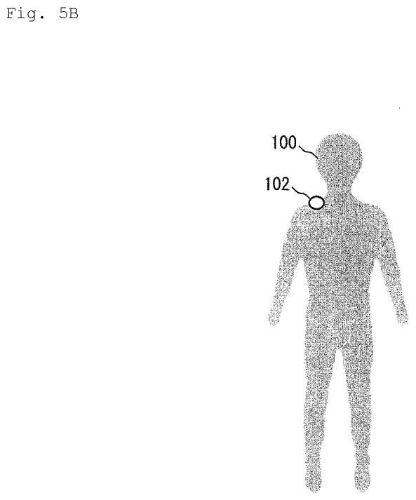

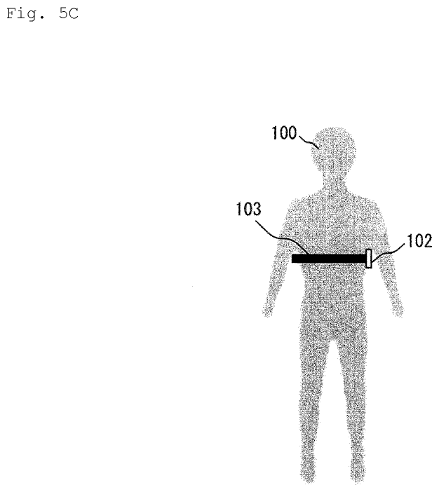

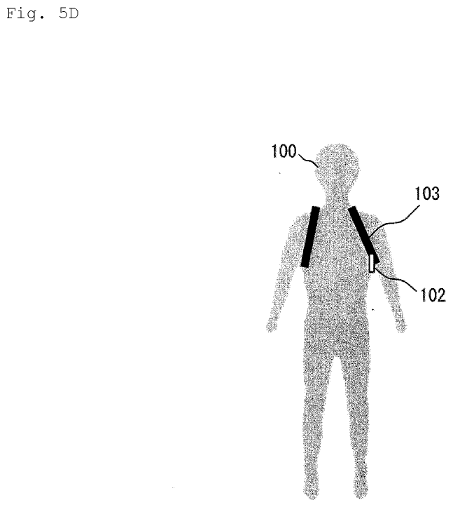

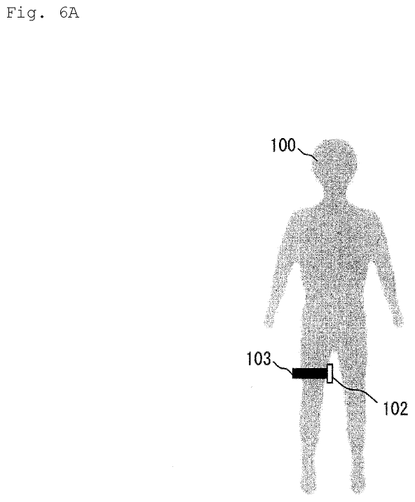

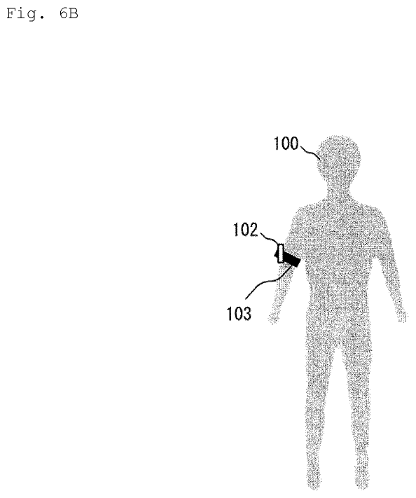

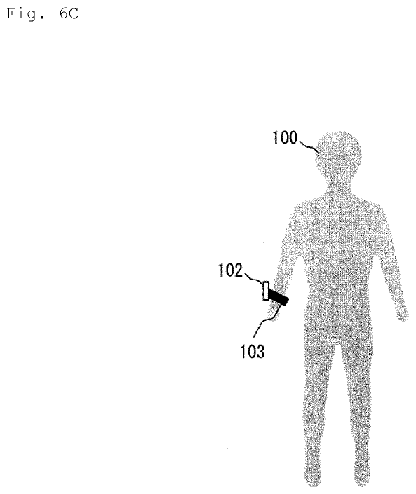

As shown in FIGS. 5A to 5D and FIGS. 6A to 6C, the temperature measuring device 102 can be mounted to various parts of the living body 100, but in any case, it is desirable that the temperature measuring device 102 be in direct contact with the skin of the living body 100.

In the example shown in FIG. 5A, the temperature measuring device 102 is stuck to the forehead of the living body 100, using a double-sided tape for the living body. In the example of FIG. 5B, the temperature measuring device 102 is stuck to the position of the clavicle of the living body 100.

In the examples of FIGS. 5C and 5D, the temperature measuring device 102 is mounted on the armpit part of the living body 100, using a stretchable band 103. In the example of FIG. 6A, the temperature measuring device 102 is mounted on the thigh of the living body 100. In the example shown in FIG. 6B, the temperature measuring device 102 is mounted on the upper arm of the living body 100. In the example of FIG. 6C, the temperature measuring device 102 is mounted on the wrist of the living body 100. Although the band 103 is used in the examples of FIGS. 5C, 5D, and 6A to 6C, the temperature measuring device 102 may be mounted on the living body 100 by the pressure of the compression wear worn by the living body 100.

The proportional coefficient Rₛₑₙₛₒᵣ used for estimating the deep body temperature can be obtained in advance by measuring the tympanic membrane temperature the rectal temperature, the axillary temperature and the like by other sensors as described above. When the axillary temperature is used as the reference temperature for obtaining the proportional coefficient Rₛₑₙₛₒᵣ, the temperature when a commercially available clinical thermometer is mounted on the axillary part of the living body 100 for about several minutes and the temperature Tₛₖᵢₙ and Tₜ become approximately the same as each other may be used as the reference temperature.

In this example, when the sensor unit 1 is formed into a cylindrical shape having a diameter D of 30 mm and a thickness t of 4 mm, the heat conductor 10 may be made of a material with a thermal conductivity of 1 W/m² or more. When the heat conductor 10 is formed in a truncated cone shape, the diameter d₁ of the through-hole 16 is about 8 mm, the diameter d₂ of the outer edge of the heat conductor 10 is about 16 mm to 30 mm, the thickness t₂ of the heat conductor 10 is 1 mm or more, the covering materials 11 and 14 are made of a material with a thermal conductivity of about 0.2 W/m², the housing 15 is made of the same material as the covering materials 11 and 14, the thickness of the housing 15 is about 0.5 mm and the interval between the temperature sensors 12 and 13 is about 2 mm, the deep body temperature T_{body} can be measured with an accuracy of approximately +0.1° C. When the diameter D of the sensor unit 1 is set to 26 mm or less, it is necessary to set the thermal conductivity of the heat conductor 10 to 10 W/m² or more.

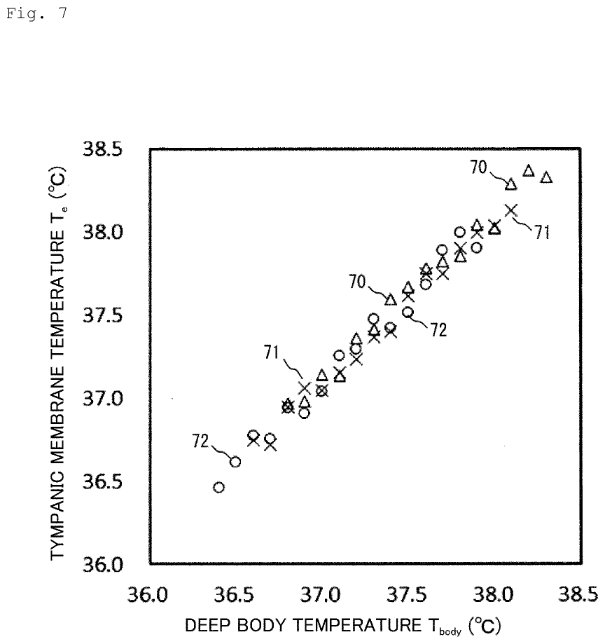

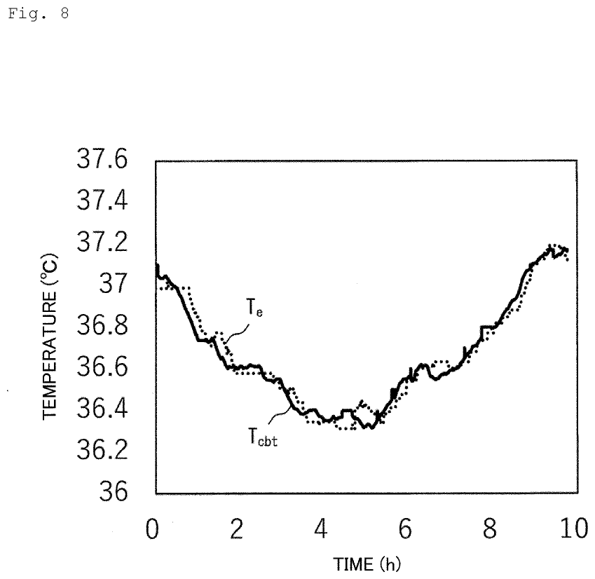

FIG. 7 shows a deep body temperature T_{body} estimated by mounting the temperature measuring device 102 of this example on the forehead of the living body 100, and a deep body temperature (tympanic membrane temperature) Tₑ measured by a tympanic membrane thermometer for comparison. Reference numerals 70, 71 and 72 of FIG. 7 show results for different living bodies 100. FIG. 8 shows changes in the deep body temperature T_{body} and the tympanic membrane temperature Tₑ estimated by the temperature measuring device 102 with time. According to FIGS. 7 and 8, it can be seen that the estimation result close to the tympanic membrane temperature Tₑ is obtained by this embodiment.

### Second Example

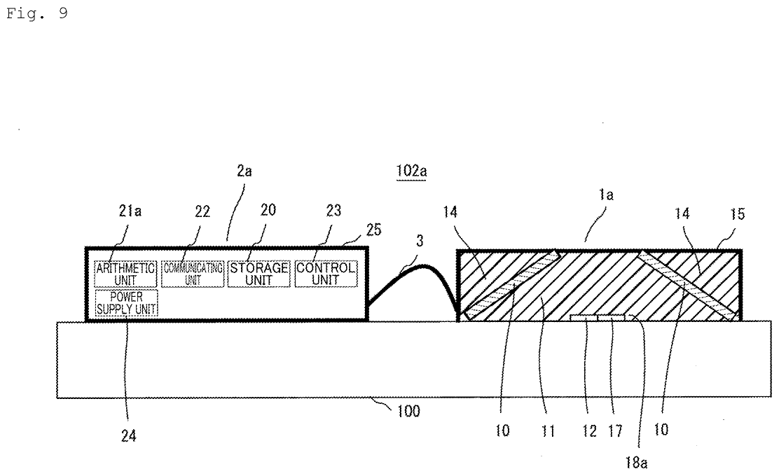

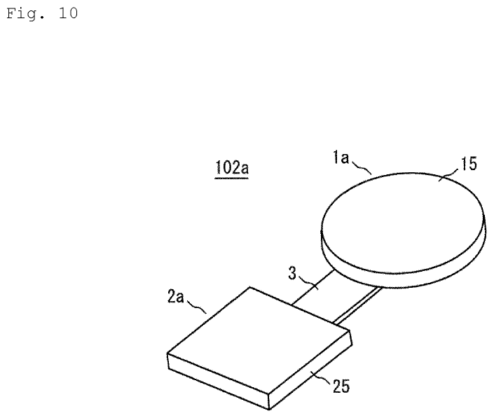

Next, a description will be given of a second example of the present invention. FIG. 9 is a diagram showing the configuration of a temperature measuring device according to a second example of the present invention, and FIG. 10 is an external view of the temperature measuring device. A temperature measuring device 102a of this example is made up of a sensor unit 1a and an electronic circuit unit 2a.

In this example, instead of the temperature sensor 13 of the sensor unit 1 of the first example, a heat flux sensor 17 is provided on the surface of the covering material 11 of the sensor unit 1a facing the living body 100. The temperature sensor 12 and the heat flux sensor 17 constitute a detection unit 18a that measures the magnitude of the heat flow. The other constitution of the sensor unit 1a is the same as that of the sensor unit 1.

The electronic circuit unit 2a includes a storage unit 20, an arithmetic unit 21a, a communication unit 22, a control unit 23, a power supply unit 24, and a housing 25.

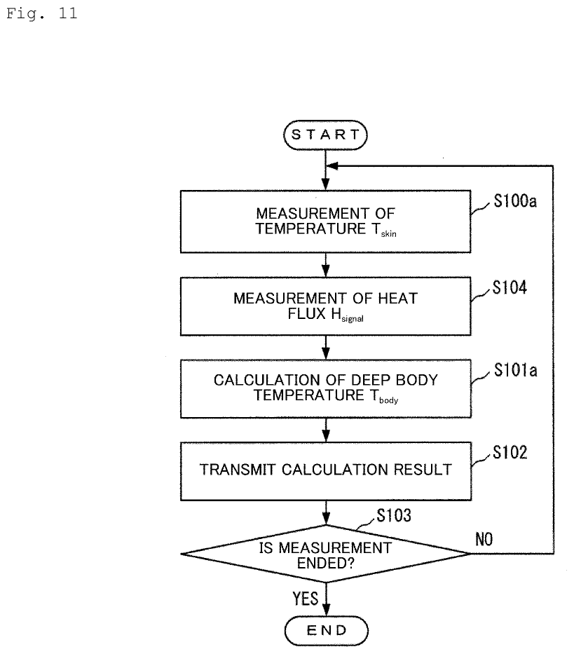

FIG. 11 is a flowchart explaining the operation of the temperature measuring device 102a of this example. The temperature sensor 12 measures the temperature Tₛₖᵢₙ of the skin surface of the living body 100, as in the first example (step S100a of FIG. 11).

The heat flux sensor 17 measures a heat flux H_{signal} flowing into the sensor unit 1a from the living body 100 (step S104 of FIG. 11). Measured data of the temperature sensors 12, and the heat flux sensor 17 is stored in the storage unit 20 once.

As in the first example, the proportional coefficient Rₛₑₙₛₒᵣ is stored in advance in the storage unit 20. The arithmetic unit 21a calculates the deep body temperature T_{body} of the living body 100 by, for example, equation (1) on the basis of the temperature Tₛₖᵢₙ, the heat flux H_{signal} and the proportional coefficient Rₛₑₙₛₒᵣ (step S101a of FIG. 11).

The communication unit 22 transmits data of the deep body temperature T_{body} to the external terminal (step S102 of FIG. 11).

The temperature measuring device 102a performs the processing of the steps S100a, S104, S101a, and S102, for example, until a user instructs the end of measurement (YES in step S103 of FIG. 11), every fixed time.

In this way, in this example, the same effects as those of the first example can be obtained.

### Third Example

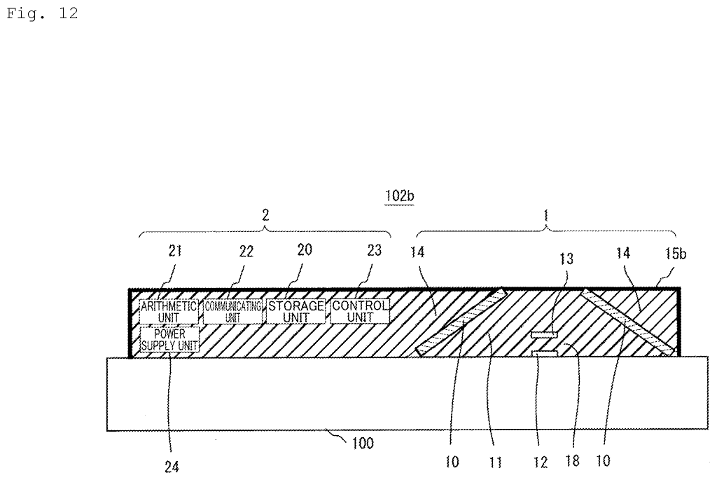

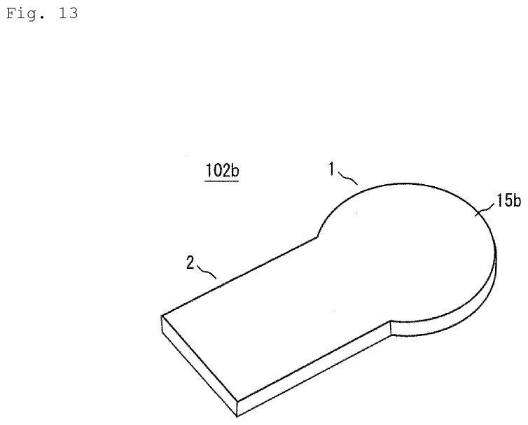

Next, a description will be given of a third example of the present invention. FIG. 12 is a diagram showing a configuration of a temperature measuring device according to a third embodiment of the present invention, and FIG. 13 is an external view of the temperature measuring device. In the temperature measuring device 102b of this example, the sensor unit 1 and the electronic circuit unit 2 are housed in the same housing 15b. In this example, the electronic circuit unit 2 is provided inside the covering material 14 that covers the heat conductor 10 of the sensor unit 1.

The sensor unit 1a of the second example may be provided instead of the sensor unit 1, and the electronic circuit unit 2a of the second example may be provided instead of the electronic circuit unit 2. The operation of the temperature measuring device 102b is the same as that of the first example or the second example.

### Fourth Example

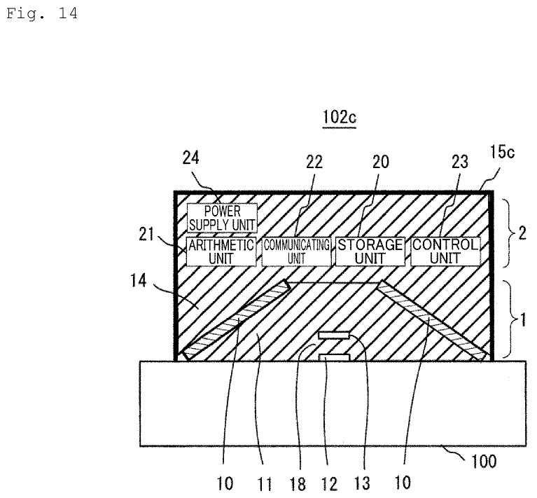

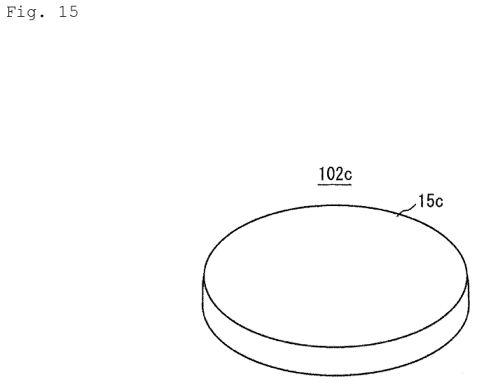

Next, a fourth example of the present invention will be described. FIG. 14 is a diagram showing a configuration of a temperature measuring device according to a fourth embodiment of the present invention, and FIG. 15 is an external view of the temperature measuring device. In a temperature measuring device 102c of this example, then electronic circuit unit 2 is provided on the sensor unit 1, and the sensor unit 1 and the electronic circuit unit 2 are stored in the same housing 15c. In this example, the electronic circuit unit 2 is provided inside the covering material 14 that covers the heat conductor 10 of the sensor unit 1.

According to this example, the installation area of the temperature measuring device 102c can be reduced. The sensor unit 1a of the second example may be provided instead of the sensor unit 1, and the electronic circuit unit 2a of the second example may be provided instead of the electronic circuit unit 2. The operation of the temperature measuring device 102c is the same as that of the first example or the second example.

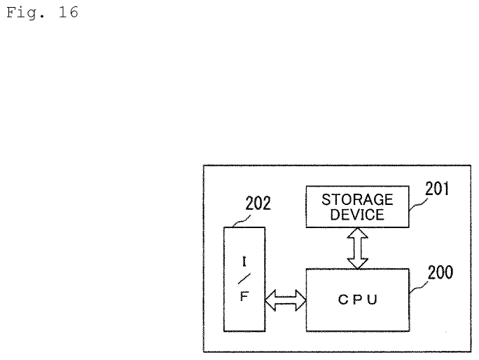

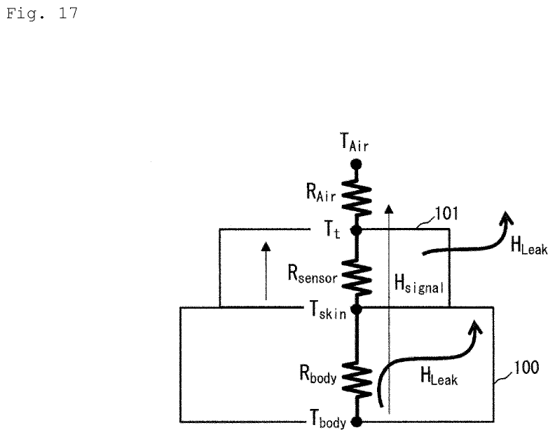

The storage unit 20, the arithmetic units 21 and 21a, the communication unit 22, and the control unit 23 explained in the first to fourth examples can be realized by a computer having a central processing unit (CPU), a storage device, and an interface, and a program that controls these hardware resources. FIG. 16 shows a configuration example of the computer.

The computer includes a CPU 200, a storage device 201, and an interface device (I/F) 202. The temperature sensors 12 and 13, the heat flux sensor 17, the hardware of the communication unit 22, and the like are connected to the I/F 202. In such a computer, program for realizing the temperature measuring method of the present invention is stored in the storage device 201. The CPU 200 executes the processing described in the first to fourth examples in accordance with the program stored in the storage device 201.

### INDUSTRIAL APPLICABILITY

The present invention can be applied to techniques for non-invasively measuring the internal temperature of a living body.

### REFERENCE SIGNS LIST

1, 1a Sensor unit
2, 2a Electronic circuit unit
10 Heat conductor
11, 14 Covering material
12, 13 Temperature sensor
15, 15b, 15c, 25 Housing
17 Heat flux sensor
18, 18a Detection unit
20 Storage unit
21, 21a Arithmetic unit
22 Communication unit
23 Control unit
24 Power supply unit
102, 102a to 102c Temperature measuring device

1-6. (canceled)

7. A temperature measuring device, comprising:
a sensor configured to measure a magnitude of a heat flow transmitted from a living body; and
an electronic circuit configured to calculate an internal temperature of the living body based on the magnitude of the heat flow measured by the sensor,
wherein the sensor includes:
a heat conductor of a hollow structure disposed so that a peripheral edge part is configured to be in contact with the living body,
a first covering material configured to fill a space between the living body and the heat conductor,
a detector on the first covering material configured to measure the magnitude of the heat flow transmitted from the living body, and
a second covering material covering the heat conductor.

8. The temperature measuring device according to claim 7, wherein the electronic circuit is provided inside the second covering material beside the sensor.

9. The temperature measuring device according to claim 7, wherein the electronic circuit is inside the second covering material on the sensor.

10. The temperature measuring device according to claim 7, further comprising:
a housing covering the second covering material on an outer side.

11. The temperature measuring device according to claim 7, wherein the detector is configured to include:
a first temperature sensor on a surface of the first covering material configured to face the living body and to measure a temperature of a surface of the living body, and
a second temperature sensor configured to measure a temperature inside the first covering material immediately above the first temperature sensor,
wherein the electronic circuit is configured to calculate an internal temperature of the living body based on measurement results of the first and second temperature sensors.

12. The temperature measuring device according to claim 7, wherein the detector is configured to include:
a temperature sensor on a surface of the first covering material configured to face the living body and to measure the temperature of a surface of the living body, and
a heat flux sensor on the surface of the first covering material configured to face the living body and to measure the heat flux flowing from the living body into the sensor,
wherein the electronic circuit is configured to calculate an internal temperature of the living body based on measurement results of the temperature sensor and the heat flux sensor.

13. A system, comprising:
a sensor configured to measure a specific physical parameter selected from the group consisting of temperature, pressure, humidity, motion, and light intensity; and
a detector coupled to the sensor, the detector being configured to analyze the measurement from the sensor by comparing the measurement to a predetermined threshold and to generate an output signal that indicates whether the measurement exceeds the predetermined threshold, wherein the detector includes a processor with a memory storing the predetermined threshold and an algorithm for analysis of the sensor measurement.

14. The system of claim 13, wherein the sensor is selected from the group consisting of a temperature sensor, a pressure sensor, a humidity sensor, a motion sensor, and an optical sensor.

15. The system of claim 13, wherein the detector includes a processor programmed to compare the measurement from the sensor to a predetermined threshold.

16. The system of claim 15, wherein the detector is further configured to trigger an alert if the measurement exceeds the predetermined threshold.

17. The system of claim 13, further comprising a transmitter connected to the detector and configured to transmit the output to an external device.

18. The system of claim 17, wherein the transmitter utilizes at least one of a wired and a wireless communication protocol.

19. The system of claim 13, wherein the detector is integrated with the sensor in a single housing.

20. The system of claim 13, wherein the detector is configured to perform real-time analysis of the measurement from the sensor.

21. A method, comprising:
measuring a physical parameter with a high-precision sensor capable of detecting minute changes in the physical parameter, wherein the physical parameter is selected from the group consisting of temperature, pressure, humidity, motion, and light intensity;
analyzing the measurement with a detector that is coupled to the sensor, wherein the analysis includes comparing the measurement to a predetermined threshold stored within a memory of the detector;
generating an output signal based on the analysis, wherein the output signal is configured to indicate whether the measurement exceeds the predetermined threshold; and
triggering an alert if the measurement is determined to exceed the threshold.

22. The method of claim 21, further comprising transmitting the output signal to an external device using a transmitter connected to the detector.

23. The method of claim 21, wherein the physical parameter is selected from the group consisting of temperature, pressure, humidity, motion, and light intensity.
